(19) United States
(12) Patent Application Publication
(10) Pub. No.: US 2008/0091067 A1
(43) Pub. Date: Apr. 17, 2008

(54) MULTI-LUMEN CATHETER AND ENDOSCOPIC METHOD

(76) Inventor: Mike Dollar, Garland, TX (US)

Correspondence Address: CARSTENS & CAHOON, LLP, P O BOX 802334, DALLAS, TX, 75380, US

(21) Appl. No.: 11/549,938

(22) Filed: Oct. 16, 2006

(51) Int. Cl. A61B 1/05

(52) U.S. Cl. 600/116; 606/192

(57) ABSTRACT

The present invention is directed towards a multi-lumen catheter with an expandable device, such as a balloon, incorporated into its surface. The multi-lumen catheter also has an opening in its shaft that allows an imaging instrument to exit the shaft and view the expandable device built into its surface. The present invention also covers an endoscopic surgical method utilizing the multi-lumen catheter.

### FIELD OF THE INVENTION

This invention relates to multi-lumen catheters and methods for using such catheters for treating paranasal sinuses.

### BACKGROUND OF THE INVENTION

In order to fully understand this invention, it is important to consider the anatomy of the sinus system. The sinus system consists of many different pathways, called ducts or ostia, which allow mucus, air and other substances to drain and flow through the system. Inflammation can occur in the tissues that make up the ducts and ostia, causing them to swell and block the normal flow. Inflammation may be caused by allergies, noxious agents, nasal polyps, and other factors. Over time there can be a pathologic increase in inflamed tissue causing permanent disruption in the flow through the sinus system. Obstruction of the narrow ducts and ostia between the paranasal sinuses and nasal cavity develops, resulting in a vicious cycle of increased secretions, edema and ultimately complete blockage of the sinus pathways. The state of chronic sinus inflammation is called sinusitis.

Treatment with antibiotics, corticosteroids in nasal sprays or systematically, and antihistamines may result in effective resolution of sinusitis. However, some patients become resistant to medical treatment and surgery becomes necessary. Endoscopic sinus surgery is performed from an intranasal approach, thus eliminating the need for external incisions. A type of minimally invasive surgery called balloon catheterization or sinuplasty involves placing an expandable device, such as a deflated balloon, inside the clogged sinus pathways and inflating the balloon in order to open the clogged pathway. A fluoroscope or image guided surgery system is required to place the balloon in the proper position. Since space inside the nasal cavity is limited, use of standard 4 millimeter diameter endoscopes for direct visual placement assistance is prohibitive. This type of surgery has also been used to open clogged pathways in other body systems, including in the vascular system, the urinary tract, and the lacrimal system. Some catheters are “steerable catheters” in that they incorporate a means that allows surgeons to direct the tip in at least one direction, thereby allowing the surgeon to “steer” the tip of the catheter to the region of interest inside the body. Steerable catheters typically contain one or two small lumens that run the length of the catheter and house steering wires. The steering wires are attached to the tip of the catheter so that pulling on one of the steering wires will deflect the tip in the direction of the steering wire. The present invention will work with either the “steerable” type of catheter or the “non-steerable” type of catheter.

### SUMMARY OF THE INVENTION

The present invention is thus directed towards a multi-lumen catheter and endoscopic method of surgery utilizing the multi-lumen catheter to perform balloon catheterization.

The first embodiment of the present invention is a four lumen catheter with an expandable device, such as a balloon, built directly into the tip of the catheter. Each of the four lumens is designed to be useful for different functions. One lumen is dedicated to inflating and deflating the balloon. A second lumen is used to insert a traditional balloon catheter into the obstructed pathway. A third lumen carries an endoscope or fiberscope, which allows the surgeon to guide the catheter to the correct location. The fourth lumen is designed to allow the surgeon to remove the fiberscope out of the third lumen and run it back through the fourth lumen. The fourth lumen, however, allows the fiberscope to escape from the catheter shaft before it gets to the tip, thereby giving the surgeon a better view of the balloon on the tip of the multi-lumen catheter inflating.

The endoscopic surgical method utilizing the first embodiment of the present invention uses all four lumens. First, an endoscope is placed inside the third lumen in such a way that the surgeon can view where the tip of the multi-lumen catheter is traveling. Next, the surgeon inserts the multi-lumen catheter into the sinus cavity and guides the tip of the multi-lumen catheter towards the obstruction. Once the tip of the multi-lumen catheter is in place, a traditional balloon catheter is inserted through the second lumen into the obstructed pathway, inflated, deflated, and removed. Next, the tip of the multi-lumen catheter is inserted into the obstructed pathway. The fiberscope is removed from the third lumen and inserted into the fourth lumen until it travels outside the multi-lumen catheter shaft behind the balloon on the tip of the multi-lumen catheter. The balloon on the tip of the multi-lumen catheter is then inflated and deflated while the surgeon watches the progress and makes adjustments as needed.

The second embodiment of the present invention is a three lumen catheter with an expandable device, such as a balloon, built directly into the tip of the catheter. Each of the three lumens is designed to be useful for different functions. The first lumen is dedicated to inflating and deflating the balloon. The second lumen carries an endoscope or fiberscope, which allows the surgeon to guide the multi-lumen catheter to the correct location. The second lumen in the second embodiment has a portion of the shaft that allows the surgeon to partially retract the fiberscope into the second lumen and make it travel outside the catheter shaft behind the balloon built into the tip of the multi-lumen catheter. The third lumen is used to insert a traditional balloon catheter into the obstructed pathway.

The endoscopic surgical method utilizing the second embodiment of the present invention uses all three lumens. First, an endoscope is placed inside the second lumen in such a way that the surgeon can view where the tip of the multi-lumen catheter is traveling. Next, the surgeon inserts the multi-lumen catheter into the sinus cavity and guides the tip of the multi-lumen catheter towards the obstruction. Once the tip of the multi-lumen catheter is in place, a traditional balloon catheter is inserted through the third lumen into the obstructed pathway, inflated, deflated, and removed. Next, the tip of the multi-lumen catheter is inserted into the obstructed pathway. The fiberscope is partially retracted inside the second lumen and guided through an opening in the multi-lumen catheter shaft and outside shaft behind the balloon on the tip of the multi-lumen catheter. The balloon on the tip of the multi-lumen catheter is then inflated and deflated while the surgeon watches the progress and makes adjustments as needed.

Both the devices and the methods of the present invention are novel over the prior art in several respects. The present invention allows the surgeon to use two balloons of different size to produce better results from the endoscopic surgery. The first balloon is a traditional balloon catheter that opens up the obstructed pathway to a certain degree. The balloon built into the tip of the multi-lumen catheter is larger in diameter, so it enlarges the pathway more than the traditional balloon catheter was able to. The present invention also eliminates the need for the surgeon to navigate the sinus system and direct a balloon catheter to the affected region twice. Instead, the larger balloon on the tip of the multi-lumen catheter is already very close to the affected region when the smaller balloon is removed and can be used immediately. The present invention also allows the surgeon to have a complete view of the entire process by allowing the endoscope to travel outside the catheter shaft directly behind the balloon.

### BRIEF DESCRIPTION OF THE DRAWINGS

A more complete understanding of the method of the present invention may be had by reference to the following detailed description when taken in conjunction with the accompanying drawings, wherein:

FIG. 1 is a cross-sectional view of the present invention inside the nasal cavity in the process of widening an obstructed pathway;

FIG. 2 is a cross-sectional view of the present invention inside the nasal cavity in the process of widening an obstructed pathway;

FIG. 3 is a cross-sectional view of the present invention inside the nasal cavity in the process of widening an obstructed pathway;

FIG. 4 is a cross-sectional view of the present invention inside the nasal cavity in the process of widening an obstructed pathway;

FIG. 5 is a cross-sectional view of the balloon tipped section of the multi-lumen catheter shaft of the first embodiment of the present invention depicting the opening through which the balloon is inflated and deflated;

FIG. 6 is a cross-sectional view of the balloon tipped section of the multi-lumen catheter shaft of the second embodiment of the present invention depicting the opening through which the balloon is inflated and deflated;

FIG. 7 is an angled outside view of the multi-lumen catheter shaft of the first embodiment of the present invention depicting the opening through which a fiberscope is directed outside the catheter shaft;

FIG. 8 is an angled outside view of the multi-lumen catheter shaft of the second embodiment of the present invention depicting the opening through which a fiberscope is directed outside the catheter shaft;

FIG. 9 is a flowchart depicting the surgical method that utilizes the first embodiment of the present invention;

Where used in the various figures of the drawing, the same numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

The present invention is directed towards a multi-lumen catheter and method for using it to perform endoscopic surgery.

Referring initially to FIG. 7, therein is depicted the first embodiment of the present invention. The first embodiment is a multi-lumen catheter 10 containing four lumens. It is understood that more lumens can be included in the catheter as needed. A lumen is a hollow, tubular portion of the multi-lumen catheter 10, approximately circular in cross section. The diameter of the lumens can vary from about 0.2 millimeters to about 1.3 millimeters. The overall diameter of the catheter shaft is approximately 4 millimeters. The multi-lumen catheter shaft is flexible and has a proximal end and a distal end. The proximal end is not depicted in because it is not important to the claimed invention. The distal end is the tip of the multi-lumen catheter and is inserted into the surgical patient's body. The distal end of the multi-lumen catheter incorporates an inflatable balloon 30 element into its surface.

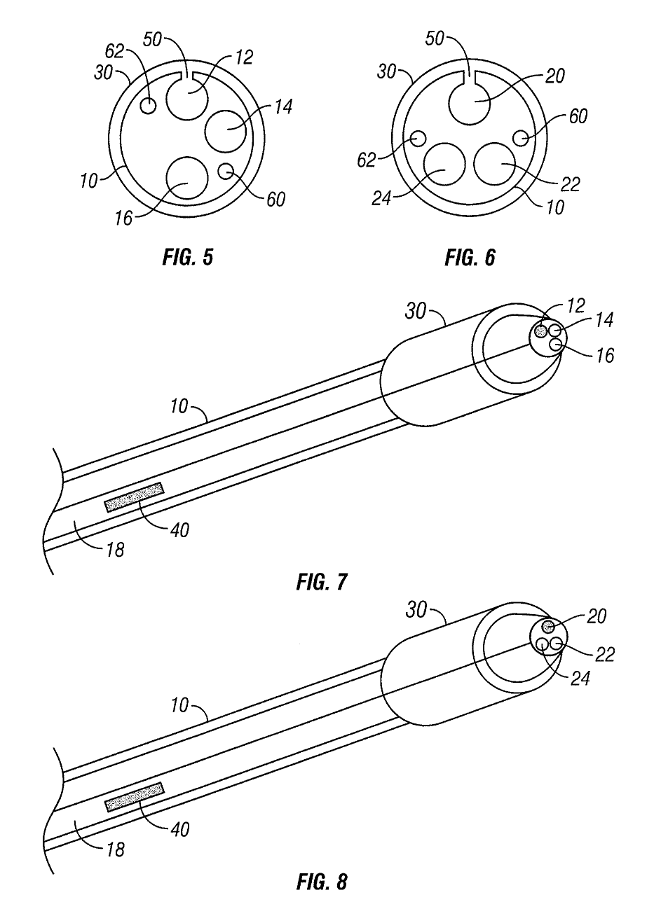

Referring now to FIG. 5, therein is depicted a cross section of the first embodiment of the multi-lumen catheter of the present invention. The cross-section in FIG. 5 depicts the balloon surface 30, the multi-lumen catheter shaft surface 10, the first lumen 12, the second lumen 14, and the third lumen 16. The fourth lumen 18 is depicted in FIG. 7, but not in FIG. 5 because the fourth lumen 18 terminates at the surface of the multi-lumen catheter 10 behind the balloon 30. FIG. 5 also depicts two optional lumens 60 and 62 that are smaller in circumference than the four main lumens of the present invention. The two optional lumens 60 and 62 are used to house the steering wires used in steerable catheters. The first lumen 12 in FIG. 5 is used to inflate and deflate the balloon 30 at the tip by transmitting fluid under pressure into and out of the balloon 30 through the hole 50 in the shaft of the multi-lumen catheter connecting the inside of the first lumen 12 to the inside of the balloon 30.

Referring back to FIG. 7, the first lumen 12 is sealed at the tip of the multi-lumen catheter 10. The second lumen 14 is open at the tip and used to insert an instrument into the patient's body, such as a traditional balloon catheter or other medical device used by those skilled in the art. The third lumen 16 is also open at the tip and is used to carry an imaging instrument, such as an endoscope of fiberscope. The term “imaging instrument” as used herein in either the specification or the claims is meant to encompass fiberscopes, endoscopes, and all other long, thin, flexible viewing instruments used by surgeons and those skilled in the art during endoscopic surgery that allow the user to view the interior of the human body. The fourth lumen 18 ends in an opening 40 in the shaft of the multi-lumen catheter located behind the balloon 30 tip. The surgeon uses the fourth lumen 18 to place the imaging instrument in a position behind the balloon 30 tip so it can capture a clear view of the inflation of the balloon 30 tip.

Referring next to FIG. 8, therein is depicted the second embodiment of the present invention. The second embodiment is a multi-lumen catheter 10 with three lumens, a proximal end, and a distal end. The first lumen 20 sealed at the tip of the multi-lumen catheter 10 and is used to inflate and deflate the balloon 30 just as it is used in the first embodiment. Likewise, the second lumen 22 is open at the tip and used to insert a traditional balloon catheter or other instrument used by one skilled in the art. The third lumen 24 of the second embodiment is open at the tip and contains a hole 40 connecting the inside of the third lumen 24 to the surface of the catheter 10. This arrangement allows a surgeon to partially retract the imaging instrument and make it exit the hole 40 in the multi-lumen catheter shaft 10 in order to give the surgeon a clear view of the inflation of the balloon 30 tip.

Referring to FIG. 6, therein is depicted a cross section of the second embodiment of the present invention. The cross section is taken at the balloon 30 tip portion of the multi-lumen catheter 10. As with the first embodiment, the first lumen 20 is used to inflate and deflate the balloon by transmitting fluid under pressure through the hole 50 in the multi-lumen catheter surface 10 connecting the inside of the first lumen 20 to the inside of the balloon 30. FIG. 6 also depicts the second and third lumens 22 and 24, and the optional smaller lumens 60 and 62 that house the steering wires used in steerable catheters.

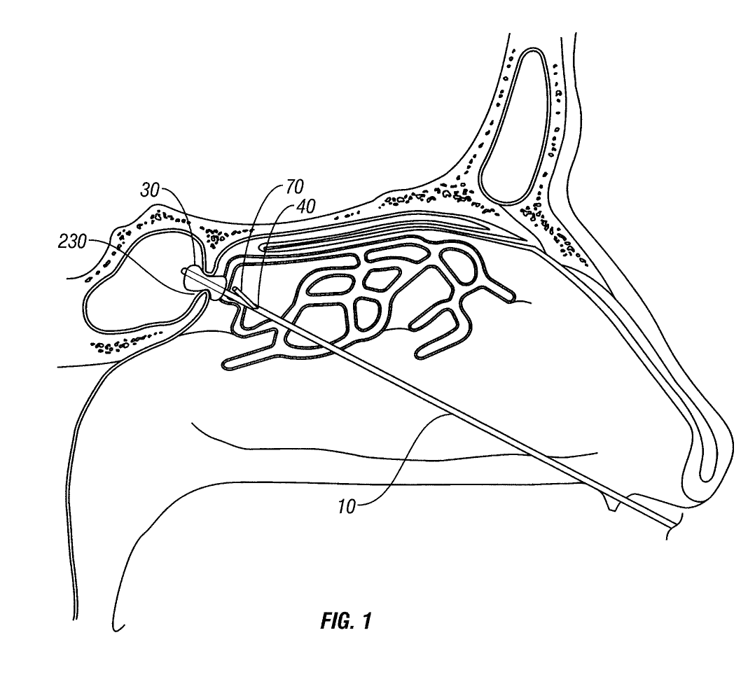

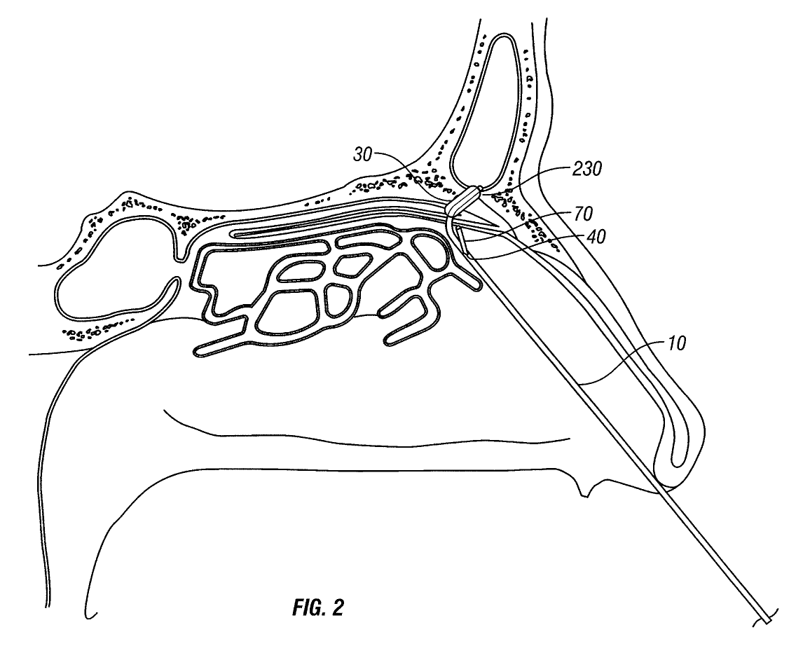

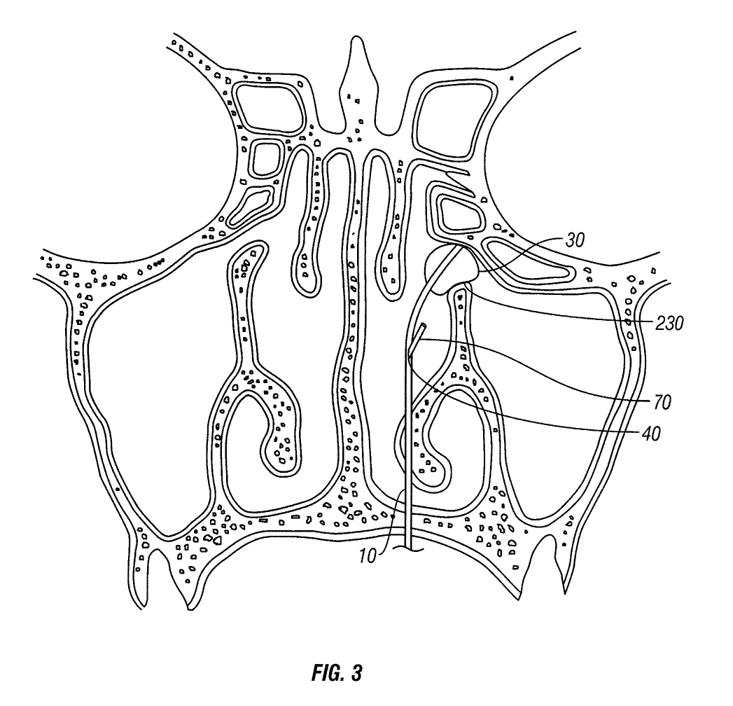

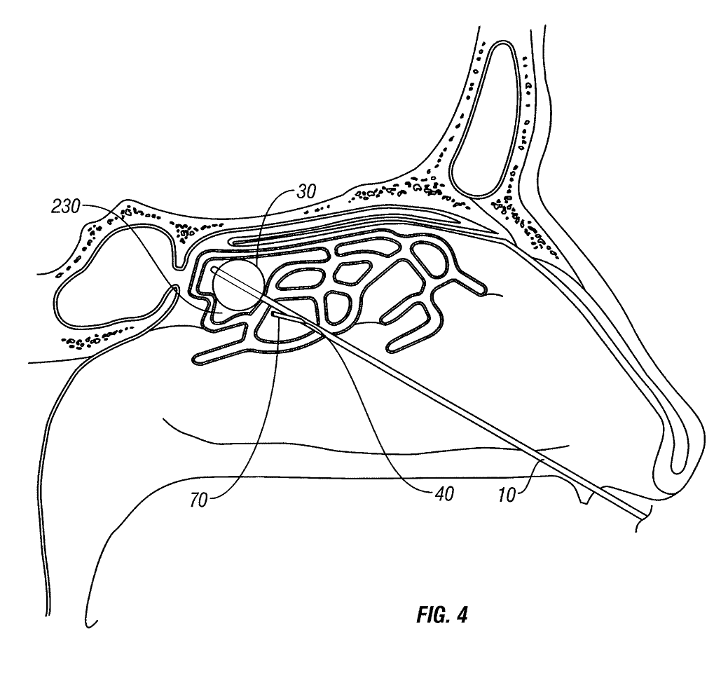

Referring next to FIG. 1, therein is depicted a cross section of the human sinus cavity, showing many of the various openings and passageways that become obstructed when a patient is suffering from chronic sinusitis. The present invention is also depicted in FIG. 1 as the balloon 30 in the tip of the multi-lumen catheter 10 is being inflated to open an obstructed passageway 230. The surgeon is watching the inflation through the imaging instrument 70 protruding from an opening 40 in the wall of the multi-lumen catheter 10. The opening is in fourth lumen of the first embodiment or the third lumen of the second embodiment. FIGS. 2, 3, and 4 similarly depict the multi-lumen catheter of the present invention in use.

The endoscopic surgical method utilizing the first embodiment of the present invention is depicted in the flow chart in FIG. 9. First, the surgeon inserts an imaging instrument into the third lumen of the multi-lumen catheter 210 to a point that allows the surgeon to look through the tip of the multi-lumen catheter. Next, the surgeon inserts the multi-lumen catheter into the system containing the obstructed fluid pathway 212 and guides the tip of the catheter to a point near the affected region. A traditional balloon catheter is then inserted through the second lumen and into the obstructed pathway 214. The balloon is inflated, deflated, and removed 216. The balloon tip of the multi-lumen catheter is then inserted into the partially opened fluid pathway 218. The surgeon then retracts the endoscope from the third lumen 220 and slides it into the fourth lumen 222 until the balloon tip of the multi-lumen catheter is in view. The surgeon can then verify the position of the balloon and make any adjustments necessary. Once the balloon tip is in the correct position, the balloon is inflated 224 and deflated 226. Because the balloon on the tip of the multi-lumen catheter is larger in diameter than a traditional balloon catheter, it is able to open the obstructed fluid pathway even wider and improve the results of the surgery. The balloon tip can optionally be inflated and deflated repeatedly 228 as needed to accomplish the task of opening the obstructed fluid pathway. The multi-lumen catheter is then retracted from the patient's body 230.

The endoscopic surgical method utilizing the second embodiment of the present invention differs only in the endoscope re-positioning step. Using the second embodiment, the surgeon only partially retracts the endoscope in the third lumen and causes it to exit the opening in the wall of the third lumen, thus giving the surgeon a clear view of the balloon tip.

We claim:

1. A multi-lumen catheter comprising:
(a) a flexible catheter shaft having a surface, a proximal end and a distal end;
(b) a first lumen sealed at the distal end of the catheter shaft;
(c) a second lumen open at the distal end of the catheter shaft;
(d) a third lumen open at the distal end of the catheter shaft;
(e) an expandable device incorporated into the surface of the catheter shaft approximately adjacent to the distal end of the catheter shaft;
(f) a fourth lumen open at the surface of the catheter shaft between the proximal end of the catheter shaft and the expandable device incorporated into the surface of the catheter shaft;
(g) an opening in the surface of the catheter shaft connecting the interior of the first lumen to the interior of the expandable device.

2. The multi-lumen catheter of claim 1 wherein the expandable device comprises a balloon.

3. The multi-lumen catheter of claim 1 additionally comprising at least one lumen adapted to housing a steering wire.

4. An endoscopic surgical method, comprising the steps of:
(a) providing a human body with a system having at least one obstructed fluid pathway;
(b) providing a balloon catheter and an imaging instrument;
(c) providing a multi-lumen catheter with a surface, a tip, an expandable device incorporated into its surface near the tip and a first, second, third and fourth lumen, with the first lumen adapted to expanding and contracting the expandable device, the second lumen adapted to carrying a surgical instrument and allowing the surgical instrument to protrude from the tip of the multi-lumen catheter, the third lumen adapted to carrying the imaging instrument and allowing the imaging instrument to protrude from the tip of the multi-lumen catheter, and the fourth lumen adapted to carrying the imaging instrument and allowing the imaging instrument to pass through the multi-lumen catheter shaft surface near the expandable device;
(d) inserting the imaging instrument in the third lumen;
(e) inserting the multi-lumen catheter into the human body;
(f) guiding the catheter tip to the obstructed fluid pathway;
(g) inserting the balloon catheter into the obstructed fluid pathway through the second lumen;
(h) inflating the balloon catheter;
(i) deflating the balloon catheter;
(j) removing the balloon catheter from the human body through the second lumen;
(k) inserting the expandable device incorporated into the surface of the multi-lumen catheter into the obstructed fluid pathway;
(l) removing the imaging instrument from the third lumen;
(m) sliding the imaging instrument through the fourth lumen until it exits an opening in the multi-lumen catheter shaft;
(n) expanding the expandable device;
(o) contracting the expandable device;
(p) removing the multi-lumen catheter from the human body.

5. The method of claim 4 comprising the additional step of repeating steps (n) and (o) at least once.

6. A multi-lumen catheter comprising:
(a) a flexible catheter shaft having a surface, a proximal end and a distal end;
(b) a first lumen sealed at the distal end of the catheter shaft;
(c) a second lumen open at the distal end of the catheter shaft;
(d) an expandable device incorporated into the surface of the catheter shaft approximately adjacent to the distal end of the catheter shaft;
(e) a third lumen open at the distal end of the catheter shaft and open at the surface of the catheter shaft between the proximal end and the expandable device incorporated into the surface of the catheter shaft;
(f) an opening in the surface of the catheter shaft connecting the interior of the first lumen to the interior of the expandable device.

7. The multi-lumen catheter of claim 6 wherein the expandable device comprises a balloon.

8. The multi-lumen catheter of claim 6 additionally comprising at least one lumen adapted to housing a steering wire.

9. An endoscopic surgical method, comprising the steps of:
(a) providing a human body with a system having at least one obstructed fluid pathway;
(b) providing a balloon catheter and an imaging instrument;
(c) providing a multi-lumen catheter with a surface, a tip, an expandable device incorporated into its surface near the tip and a first, second, and third lumen, with the first lumen adapted to expanding and contracting the expandable device, the second lumen adapted to carrying a surgical instrument and allowing the surgical instrument to protrude from the tip of the multi-lumen catheter, and the third lumen adapted to carrying the imaging instrument, allowing the imaging instrument to protrude from the tip of the multi-lumen catheter and allowing the imaging instrument to pass through the multi-lumen catheter shaft surface near the expandable device;
(d) inserting the imaging instrument in the third lumen until it protrudes from the tip of the multi-lumen catheter;
(e) inserting the multi-lumen catheter into the human body;
(f) guiding the catheter tip to the obstructed fluid pathway;
(g) inserting the balloon catheter into the obstructed fluid pathway through the second lumen;
(h) inflating the balloon catheter;
(i) deflating the balloon catheter;
(j) removing the balloon catheter from the human body through the second lumen;
(k) inserting the expandable device incorporated into the surface of the multi-lumen catheter into the obstructed fluid pathway;
(l) retracting the imaging instrument inside the third lumen;
(m) sliding the imaging instrument through the third lumen until it exits an opening in the multi-lumen catheter shaft;
(n) expanding the expandable device;
(o) contracting the expandable device;
(p) removing the multi-lumen catheter from the human body.

10. The method of claim 9 comprising the additional step of repeating steps (n) and (o) at least once.
